(19) United States
(12) Patent Application Publication
(10) Pub. No.: US 2014/0213862 A1
(43) Pub. Date: Jul. 31, 2014

(54) WAVELET-BASED SYSTEM AND METHOD FOR ANALYZING A PHYSIOLOGICAL SIGNAL

(71) Applicant: COVIDIEN LP, Boulder, CO (US)

(72) Inventors: Paul Stanley Addison, Edinburgh (GB); James Ochs, Seattle, WA (US); James Nicholas Watson, Dunfermline (GB)

(73) Assignee: Covidien LP, Boulder, CO (US)

(21) Appl. No.: 13/751,754

(22) Filed: Jan. 28, 2013

(51) Int. Cl. A61B 5/00; A61B 5/0402; A61B 5/021; A61B 5/1455; A61B 5/0205

(52) U.S. Cl. CPC A61B 5/7278; A61B 5/14551; A61B 5/0205; A61B 5/021; A61B 5/0402
USPC 600/324; 600/300; 600/485; 600/509

(57) ABSTRACT

Certain embodiments of the present disclosure provide a system and method for analyzing a physiological signal detected from an individual. The system may include a physiological signal detection module configured to detect the physiological signal of the individual, a wavelet formation module configured to form a wavelet based on the physiological signal, and a wavelet transform module configured to generate a scalogram by transforming the physiological signal with the wavelet based on the physiological signal.

### FIELD

Embodiments of the present disclosure generally relate to physiological signal processing and, more particularly, to a system and method for analyzing a physiological signal using a wavelet that is derived from, formed from, or otherwise based on the physiological signal.

### BACKGROUND

A wavelet function may be applied to a physiological signal, such as a photoplethysmograph (PPG) signal, to form a scalogram that separates the physiological signal into component parts. The component parts may be analyzed to determine one or more physical parameters, such as pulse rate.

Typically, the wavelet or wavelet function may be a mathematical function such as a Morlet wavelet, a Mexican hat wavelet, a Hermitian wavelet, a Shannon wavelet, a Beta wavelet, a Paul wavelet, or the like. However, because the wavelet function is not specific to the physiological signal itself, the resulting scalogram may contain undesired information, such as noise and interference, or otherwise be unreliable.

### SUMMARY

Certain embodiments of the present disclosure provide a system for analyzing a physiological signal detected from an individual. The system may include a physiological signal detection module configured to detect the physiological signal of the individual, a wavelet formation module configured to form a wavelet based on the physiological signal, and a wavelet transform module configured to generate a scalogram by transforming the physiological signal with the wavelet based on the physiological signal. The system may also include a physiological parameter determination module configured to determine one or more physiological parameters of the individual through an analysis of the scalogram.

The system may also include a detection sub-system in communication with the physiological signal detection module. The physiological signal detection module may be configured to receive the physiological signal from the detection sub-system. The detection sub-system may include a photoplethysmograph (PPG) sub-system, and the physiological signal may include a PPG signal. Alternatively, the detection sub-system may include a blood pressure detection sub-system, and the physiological signal may include a blood pressure signal. In another embodiment, the detection sub-system may include an electrocardiogram (EKG) sub-system, and the physiological signal may include an EKG signal.

In at least one embodiment, the wavelet formation module may be configured to form the wavelet by directly analyzing the physiological signal and shaping the wavelet to directly correlate to the physiological signal. The wavelet formation module may be configured to contain at least a portion of the physiological signal within a Gaussian window or envelope to form the wavelet. In at least one embodiment, the wavelet may include a derivative of the physiological signal. In another embodiment, the wavelet formation module may be configured to form the wavelet by using one or more shapes that are similar to one or more portions of a generic shape of the physiological signal.

Certain embodiments of the present disclosure provide a method of analyzing a physiological signal detected from an individual. The method may include detecting a physiological signal of the individual with a physiological signal detection module, forming a wavelet based on the physiological signal with a wavelet formation module, and generating a scalogram with a wavelet transform module by transforming the physiological signal with the wavelet based on the physiological signal.

Certain embodiments of the present disclosure provide a tangible and non-transitory computer readable medium that includes one or more sets of instructions configured to direct a computer to detect a physiological signal of an individual, form a wavelet based on the physiological signal, and generate a scalogram by transforming the physiological signal with the wavelet based on the physiological signal.

### BRIEF DESCRIPTION OF THE DRAWINGS

FIG. 1 illustrates a simplified block diagram of a system for determining a physiological signal, according to an embodiment of the present disclosure.

FIG. 2(a) illustrates a top plan view of a scalogram derived from a physiological signal, according to an embodiment of the present disclosure.

FIG. 2(b) illustrates an isometric top view of a scalogram derived from a physiological signal, according to an embodiment of the present disclosure.

FIG. 2(c) illustrates an exemplary scalogram derived from a signal containing two pertinent components, according to an embodiment of the present disclosure.

FIG. 2(d) illustrates a schematic of signals associated with a ridge in FIG. 2(c), and a schematic of a further wavelet decomposition of the signals, according to an embodiment of the present disclosure.

FIG. 3 illustrates a Morlet wavelet.

FIG. 4 illustrates a photoplethysmograph (PPG) signal over time, according to an embodiment of the present disclosure.

FIG. 5 illustrates a top plan view of a scalogram formed by the Morlet wavelet shown in FIG. 3 transforming the PPG signal shown in FIG. 4.

FIG. 6 illustrates an isometric view of a scalogram formed by the Morlet wavelet shown in FIG. 3 transforming the PPG signal shown in FIG. 4.

FIG. 7 illustrates a synthesized wavelet, according to an embodiment of the present disclosure.

FIG. 8 illustrates a top plan view of a scalogram formed by the synthesized wavelet shown in FIG. 7 transforming the PPG signal shown in FIG. 4, according to an embodiment of the present disclosure.

FIG. 9 illustrates an isometric view of a scalogram formed by the synthesized wavelet shown in FIG. 7 transforming the PPG signal shown in FIG. 4, according to an embodiment of the present disclosure.

FIG. 10 illustrates a simplified PPG signal over time, according to an embodiment of the present disclosure.

FIG. 11 illustrates a series of PPG signals within a Gaussian window, according to an embodiment of the present disclosure.

FIG. 12 illustrates a PPG wavelet, according to an embodiment of the present disclosure.

FIG. 13 illustrates a top plan view of a scalogram formed by the PPG wavelet shown in FIG. 12 transforming the PPG signal shown in FIG. 4, according to an embodiment of the present disclosure.

FIG. 14 illustrates an isometric view of a scalogram formed by the PPG wavelet shown in FIG. 12 transforming the PPG signal shown in FIG. 4, according to an embodiment of the present disclosure.

FIG. 15 illustrates a PPG wavelet derived from a derivative of the PPG signal shown in FIG. 4, according to an embodiment of the present disclosure.

FIG. 16 illustrates an isometric view of a photoplethysmogram system, according to an embodiment of the present disclosure.

FIG. 17 illustrates a simplified block diagram of a PPG system, according to an embodiment of the present disclosure.

FIG. 18 illustrates a blood pressure signal over time, according to an embodiment of the present disclosure.

FIG. 19 illustrates an echocardiograph (EKG) signal over time, according to an embodiment of the present disclosure.

FIG. 20 illustrates a flow chart of a method of determining one or more physiological parameters, according to an embodiment of the present disclosure.

### DETAILED DESCRIPTION

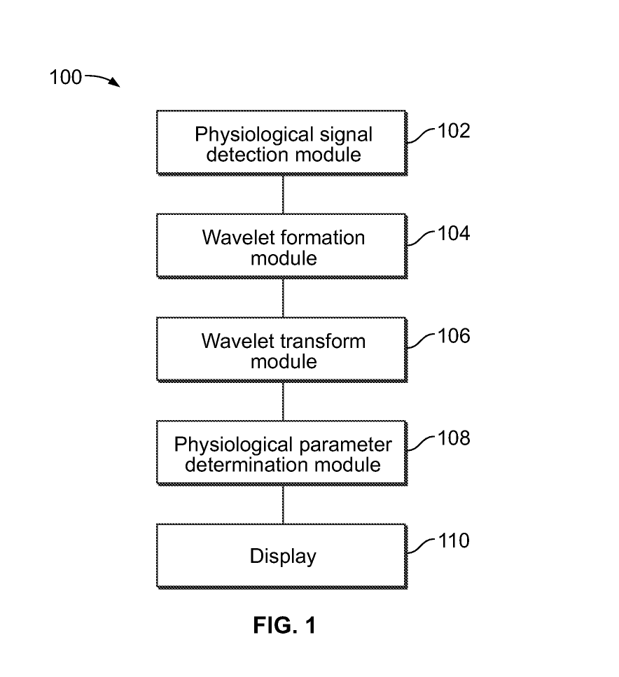

FIG. 1 illustrates a simplified block diagram of a system 100 for determining a physiological signal, according to an embodiment of the present disclosure. The system 100 may include a physiological signal detection module 102, a wavelet formation module 104, a wavelet transform module 106, a physiological parameter determination module 108, and a display 110. The system 100 is configured to detect a physiological signal, such as a PPG signal, blood pressure signal, electrocardiograph (EKG) signal, or the like, through the physiological signal detection module 102, which may be operatively connected to, and/or in communication with a detection sub-system (not shown in FIG. 1), such as pulse oximeter, blood pressure detector, EKG detector, or the like. The wavelet formation module 104 receives the physiological signal, and derives, constructs, determines, forms, or otherwise generates a wavelet based on the physiological signal itself, as opposed to using a random, arbitrary, or constant wavelet, such as a Morlet wavelet. The wavelet transform module 106 then utilizes the wavelet that is based on the physiological signal to form a scalogram, which decouples various components of the physiological signal for analysis. The physiological parameter determination module 108 then analyzes the scalogram to determine one or more physiological parameters, such as pulse rate, respiratory effort, and/or the like. Because the wavelet is based on the physiological signal itself, the resulting scalogram provides clear, accurate, and reliable information regarding the physiological parameters, as compared to a scalogram formed by using an arbitrary, random, and/or constant wavelet function. The display 110 may show one or more of the physiological signal, the wavelet, the scalogram, and/or the physiological parameters. The modules 102, 104, 106, and 108, and the display may be operatively connected to one another through circuits, cables, wireless connections, and/or the like.

The physiological signal detection module 102 may be configured to receive a physiological signal, such as a PPG or blood pressure signal, from a detection sub-system or device (not shown in FIG. 1) that is configured to detect the physiological signal of an individual. The physiological signal detection module 102 may analyze the physiological signal and display it on the display 110 as a two-dimensional waveform.

The wavelet formation module 104 analyzes the physiological signal and forms a wavelet based directly on the physiological signal itself. For example, the physiological signal may be analyzed or otherwise used to form the wavelet. The resulting wavelet may also be displayed on the display 110. The wavelet formation module 104 continually analyzes the received physiological signal. Therefore, as the physiological signal changes over time, the wavelet formation module 104 may alter the wavelet based on the changing physiological signal. Accordingly, the wavelet continually adapts to the morphology of the physiological signal over time.

The wavelet transform module 106 receives the physiological signal and the formed wavelet from the physiological signal detection module 102 and the wavelet formation module 104, respectively. The wavelet transform module 106 is configured to transform the physiological signal with the wavelet to yield a scalogram, which may also be displayed on the display 110

The physiological parameter determination module 108 is configured to analyze the scalogram to determine one or more physiological parameters, such as pulse rate, blood pressure, respiratory effort, and/or the like, which may also be shown on the display 110. As such, the physiological parameter(s) is accurate and reliable, as it is determined through use of a wavelet that is based on and directly correlates with the physiological signal itself.

The system 100 may be contained within a workstation that may be or otherwise include one or more computing devices, such as standard computer hardware. Each module 102, 104, 106, and 108 may include one or more control units, such as processing devices that may include one or more microprocessors, microcontrollers, integrated circuits, memory, such as read-only and/or random access memory, and the like.

The modules 102, 104, 106, and 108 may be integrated into a single module and contained within a single housing. Alternatively, each module 102, 104, 106, and 108 may be its own separate and distinct module, and contained within a respective housing.

The display 110 may be a cathode ray tube display, a flat panel display, such as a liquid crystal display (LCD), a light-emitting diode (LED) display, a plasma display, or any other type of monitor. As noted, the system 100 may be configured to calculate physiological parameters and to show information related to the physiological parameters on the display 110.

The system 100 may include any suitable computer-readable media used for data storage. For example, one or more of the modules 102, 104, 106, and 108 may include computer-readable media. The computer-readable media are configured to store information that may be interpreted by the modules 102, 104, 106, and 108. The information may be data or may take the form of computer-executable instructions, such as software applications, that cause a microprocessor or other such control unit within the modules 102, 104, 106, and 108 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

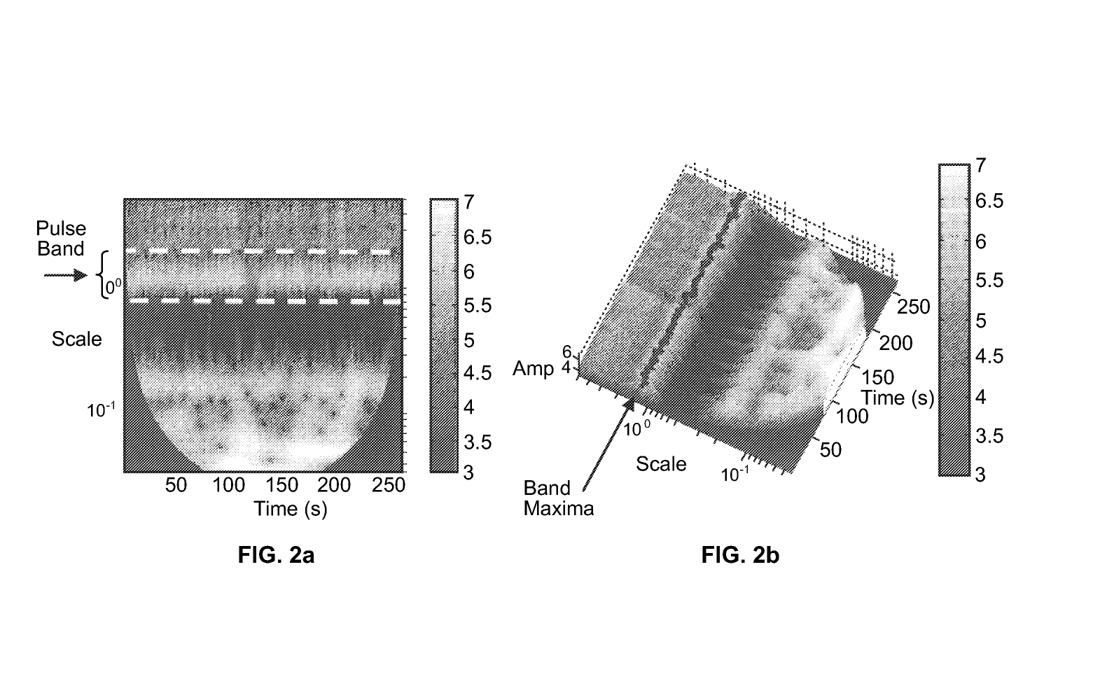

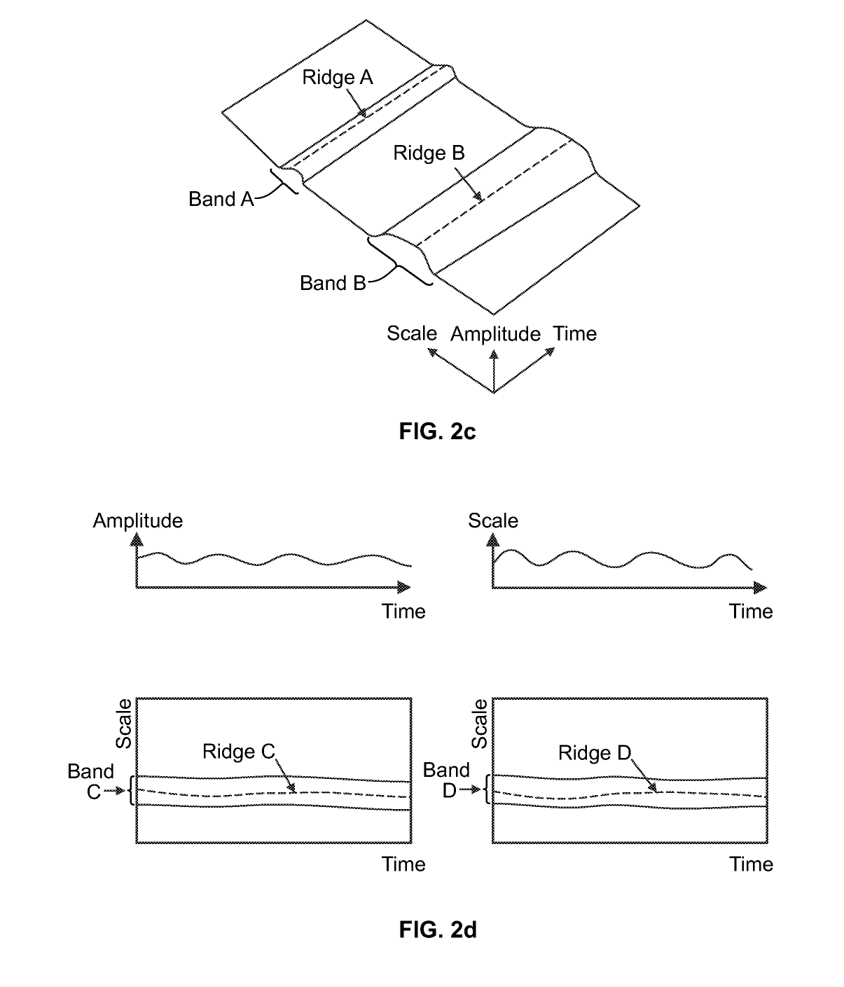

FIGS. 2(a) and 2(b) illustrate top plan and isometric views, respectively, of a scalogram derived from a physiological signal, according to an embodiment of the present disclosure. A physiological signal, such as a PPG signal, EKG signal, or a blood pressure signal, may be transformed using a wavelet transform. Information derived from the transform of the pressure signal may be used to provide measurements of one or more physiological parameters.

The wavelet transform of a signal x(t) may be defined as shown in Equation (1):

$$T ( a , b ) = \frac{1}{\sqrt{a}} {\int }_{- ∞}^{+ ∞} x ( t ) {ψ}^{*} ( \frac{t - b}{a} ) d t \qquad Equation ( 1 )$$

where ψ*(t) is the complex conjugate of the wavelet function ψ(t), a is the dilation or scale parameter of the wavelet, b is the location parameter of the wavelet, and x(t) is the signal under investigation. For example, x(t) may be a physiological signal, such as a PPG signal, a blood pressure signal, an EKG signal, or the like. The wavelet function ψ(t) may be the wavelet that transforms the physiological signal x(t) into a scalogram. The wavelet function ψ(t) may be formed from or otherwise based on the physiological signal x(t).

The transform given by Equation (1) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The wavelet transform decomposes a signal using wavelets, which are generally highly localized in time. A continuous wavelet transform may provide a higher resolution relative to discrete transforms, thus providing the ability to garner more information from signals that typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain create a series of frequency transform values in a two-dimensional coordinate system where the two dimensions may be frequency and, for example, amplitude. Wavelet transforms are further described in U.S. Pat. No. 7,944,551, entitled “Systems and Methods for a Wavelet Transform Viewer,” and U.S. Patent Application Publication No. 2010/0079279, entitled “Detecting a Signal Quality Decrease in a Measurement System,” both of which are hereby incorporated by reference in their entireties.

Referring again to Equation (1), a modulus of the transform may be defined as |T(a,b)|. As such, the energy density function of the wavelet transform (that is, the scalogram) may be rescaled as follows:

$${\vert T ( a , b ) \vert}^{*} = \frac{\vert T ( a , b ) \vert}{\sqrt{a}} \qquad Equation ( 2 )$$

The rescaled wavelet transform scalogram may be used to define ridges in wavelet space. The scalogram may be taken to include all suitable forms of rescaling including, but not limited to, the original unscaled wavelet representation, linear rescaling, any power of the modulus of the wavelet transform, or any other suitable rescaling. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”. A ridge is a locus of points of local maxima in a plane. Also, a ridge may be a path displaced from the locus of the local maxima. The rescaled wavelet transform scalogram allows for a representation in which ridges of bands on the transform surface scale directly with amplitudes of corresponding signal components. By using the rescaled transform, the direct scale relationship may take the simplified form as follows:

Aᵣ=K·Aₛ Equation (3)

where Aᵣ is the ridge amplitude, K is a constant, and Aₛ is the signal amplitude, which may be defined as the distance from peak-to-trough. For a sinusoidal signal, Equation (3) may take the form of the following:

$${A}_{r} = \frac{\sqrt[4]{π}}{2} {A}_{s} \qquad Equation ( 4 )$$

Accordingly, the ridge amplitude may be related to the signal amplitude by a constant (K) of 0.67. That is, K=\sqrt{π}/2. However, other constants may be experimentally or empirically derived, based on various factors, such as the type of wavelet transform being used.

Wavelet transform features may be extracted from the wavelet decomposition of signals. For example, wavelet decomposition of physiological signals, such as PPG or blood pressure signals, may be used to provide clinically useful information.

Pertinent repeating features in a signal give rise to a time-scale band in wavelet space or a resealed wavelet space. For example, the pulse component of a physiological pressure signal produces a dominant band in wavelet space at or around the pulse frequency. FIGS. 2(a) and 2(b) illustrate two views of an illustrative scalogram derived from a PPG signal, according to an embodiment of the present disclosure. FIGS. 2(a) and 2(b) show an example of the band caused by the pulse component in such a signal. The pulse band is located between the dashed lines in the plot of FIG. 2(a). The pulse band is formed from a series of dominant coalescing features across the scalogram. The pulse band is more clearly seen as a raised band across the transform surface in FIG. 2(b) located within the region of scales indicated by the arrow in the plot. The maxima of the pulse band with respect to the scale is the ridge. The locus of the ridge is shown as a black curve on top of the band in FIG. 2(b). By employing a suitable rescaling of the scalogram, such as that given in equation (2), the ridges found in wavelet space may be related to the instantaneous frequency of the signal. In this way, the pulse rate may be obtained from the physiological signal, such as a PPG signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. As such, both times between individual pulses and the timing of components within each pulse may be monitored and used to detect heart beat anomalies, measure arterial system compliance, or perform any other suitable calculations or diagnostics.

FIG. 2(c) illustrates an exemplary scalogram derived from a signal containing two pertinent components, according to an embodiment of the present disclosure. As noted above, pertinent repeating features in the signal give rise to a time-scale band in wavelet space or a rescaled wavelet space. For a periodic signal, the band remains at a constant scale in the time-scale plane. For many real signals, especially biological signals, the band may be non-stationary—varying in scale, amplitude, or both over time. As shown in FIG. 2(c), the two pertinent components lead to two bands in the transform space. The bands are labeled band A and band B on the three-dimensional schematic of the wavelet surface. In an embodiment, the band ridge is defined as the locus of the peak values of the bands with respect to scale. For purposes of clarity, it may be assumed that band B contains the signal information of interest. As such, band B may be referred to as the “primary band.” In addition, it may be assumed that the system from which the signal originates, and from which the transform is subsequently derived, exhibits some form of coupling between the signal components in band A and band B. When noise or other erroneous features are present in the signal with similar spectral characteristics of the features of band B, then the information within band B can become ambiguous (for example, obscured, fragmented, or missing). As such, the ridge of band A may be followed in wavelet space and extracted either as an amplitude signal or a scale signal which may be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively.

FIG. 2(d) illustrates a schematic of signals associated with a ridge in FIG. 2(c), and a schematic of a further wavelet decomposition of the signals, according to an embodiment of the present disclosure. The top plots of FIG. 2(d) illustrate a schematic of the RAP and RSP signals associated with ridge A in FIG. 2(c). Below the RAP and RSP signals are schematics of a further wavelet decomposition of the newly derived signals. The secondary wavelet decomposition allows for information in the region of band B in FIG. 2(c) to be made available as band C and band D. The ridges of bands C and D may serve as instantaneous time-scale characteristic measures of the signal components causing bands C and D. This technique, which may be referred to as secondary wavelet feature decoupling (SWFD), may allow information concerning the nature of the signal components associated with the underlying physical process causing the primary band B (shown in FIG. 2(c)) to be extracted when band B itself is obscured in the presence of noise or other erroneous signal features.

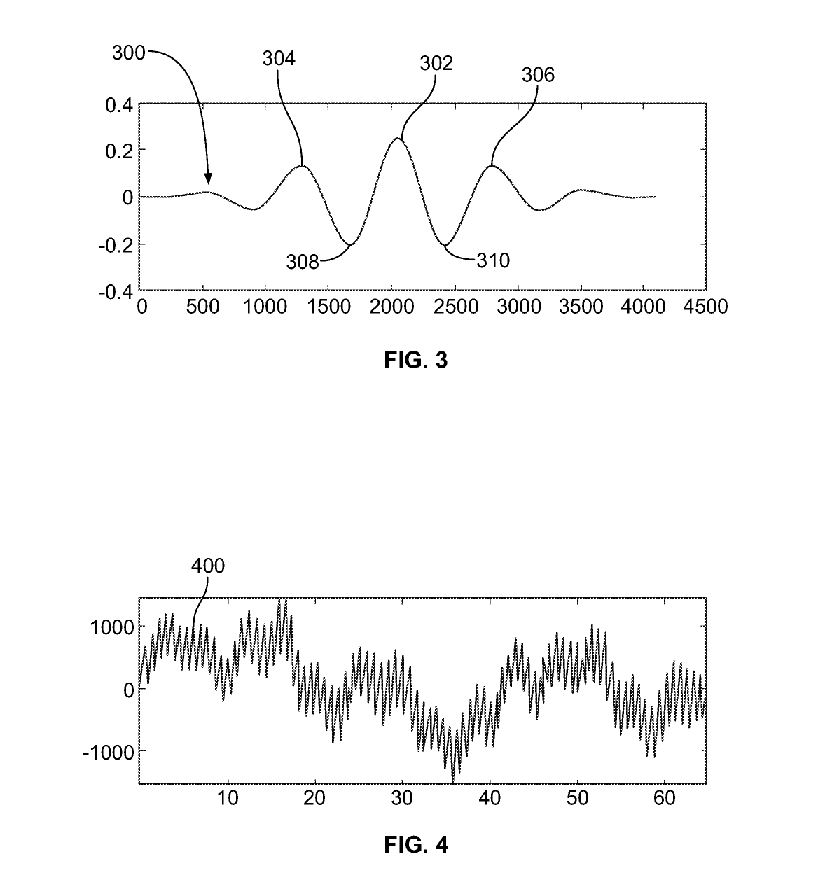

FIG. 3 illustrates a Morlet wavelet 300. As shown in FIG. 3, the Morlet wavelet is shown as a real value Morlet wavelet 300. The Morlet wavelet 300 is a wavelet that includes a complex exponential or carrier multiplied by a Gaussian window or envelope. The Morlet wavelet 300 is generally related to human perception, such as hearing and vision. The Morlet wavelet 300 may include a primary peak 302 that is preceded by a leading peak 304, and followed by a trailing peak 306. A trough 308 is disposed between the leading peak 304 and the primary peak 302, while a trough 310 is disposed between the primary peak 302 and the trailing peak 306. The primary peak 302 is typically greater in amplitude than the leading and trailing peaks 304 and 306, respectively. Each peak 302, 304, and 306 may be shaped as a generally smoothly fluctuating waveform.

FIG. 4 illustrates a PPG signal 400 over time, according to an embodiment of the present disclosure. The PPG signal 400 is an example of a physiological signal detected by the physiological signal detection module 102. However, the physiological signal may be various other types of physiological signals, such as a blood pressure signal, an EKG signal, and the like.

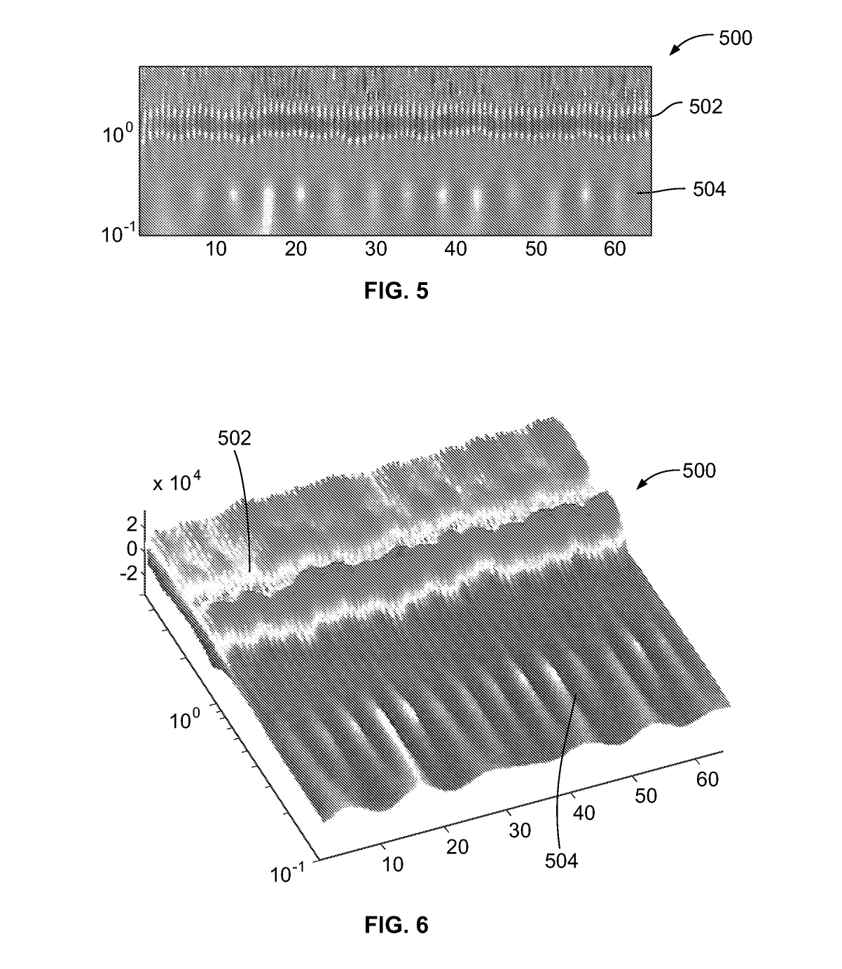

FIG. 5 illustrates a top plan view of a scalogram 500 formed by the Morlet wavelet 300 shown in FIG. 3 transforming the PPG signal 400 shown in FIG. 4. FIG. 6 illustrates an isometric view of the scalogram 500. Referring to FIGS. 3-6, the Morlet wavelet may be used as the wavelet function ψ(t), while the PPG signal is the signal x(t) in Equation (1). The Morlet wavelet 300 transforms the PPG signal 400 into the scalogram 500. As such, components of the PPG signal 400 are decoupled from one another and shown in the scalogram 500. For example, the scalogram 500 includes a main pulse band 502 and respiration components 504. The physiological parameter determination module 108 may analyze the scalogram 500, including the main pulse band 502 and the respiration components 504 to determine one or more physiological parameters, such as pulse rate, blood pressure, and the like.

However, while the Morlet wavelet 300 includes a series of smooth, waveforms, the PPG signal 400 may be irregular and include waveform portions that may be nominally sinusoidal and distinctly non-sinusoidal in shape. As such, the use of the Morlet wavelet 300 to transform the PPG signal 400 may result in a scalogram 500 that may distort certain components of the PPG signal 400. For example, certain components of the PPG signal 400 may be misrepresented, over- or under-valued, or even missing from the scalogram 500. Additionally, through use of the Morlet wavelet 300, certain undesired aspects, such as noise, may be included in the scalogram 500. Therefore, in using the Morlet wavelet 300 to transform the PPG signal 400 into the scalogram 500, the physiological parameter determination module 108 (shown in FIG. 1) may utilize additional processing and analysis in order to determine the physiological parameter(s).

Accordingly, embodiments of the present disclosure provide a system and method of forming a wavelet that may be closely and directly correlated with a physiological signal, such as the PPG signal 400, in order to provide more accurate and detailed scalograms.

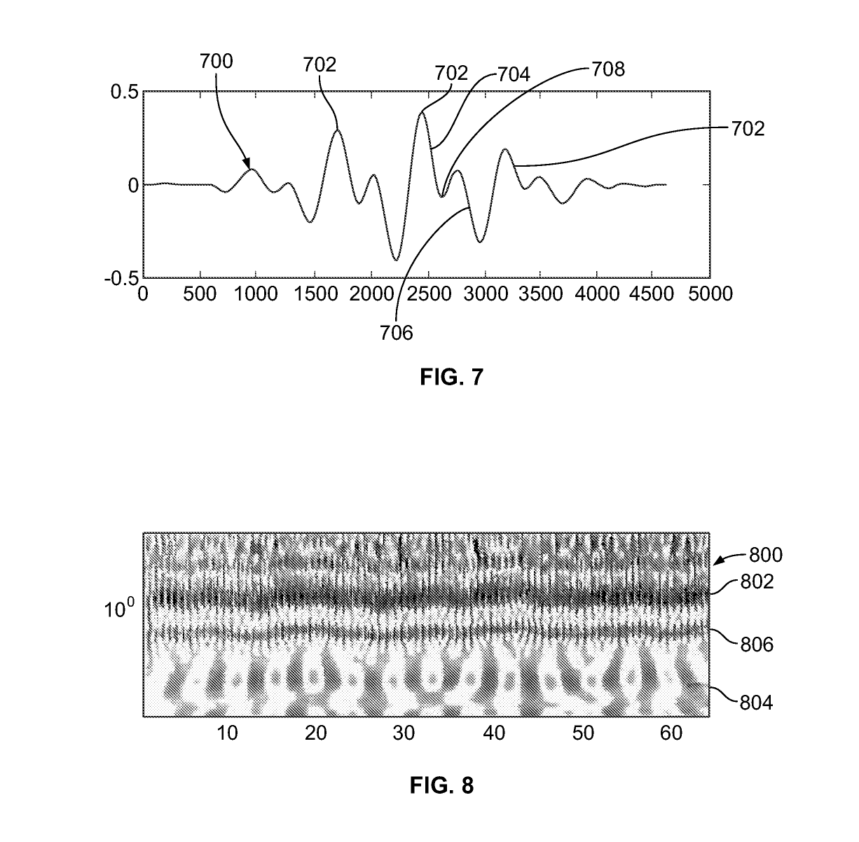

FIG. 7 illustrates a synthesized wavelet 700, according to an embodiment of the present disclosure. The synthesized wavelet 700 is similar to the Morlet wavelet 300, except, instead of smooth, regular sinusoidal peaks, the synthesized wavelet 700 includes peaks 702 having initial peak component 704 separated from a secondary peak component 706 by a notch 708. The initial peak component 704 and the secondary peak component 706 may generally be sinusoidal in shape. The secondary peak component 706 may be superimposed, added, spliced, or the like onto the initial peak component 702 to form the double-hump shape with the notch 708 shown in FIG. 7. As compared to a Morlet wavelet, the double hump shape of the peaks 702 is more closely related to a PPG signal, which may include a primary peak separated from a trailing peak separate by a dichrotic notch. Accordingly, the synthesized wavelet 700 may be used as the wavelet function ψ(t) in Equation (1) to yield a more detailed and/or accurate scalogram. In general, the wavelet formation module 104 (shown in FIG. 1) may use one or more shapes that are analogous or otherwise similar to portions of a generic shape of a physiological signal, such as a generic PPG signal, to form the synthesized wavelet 700.

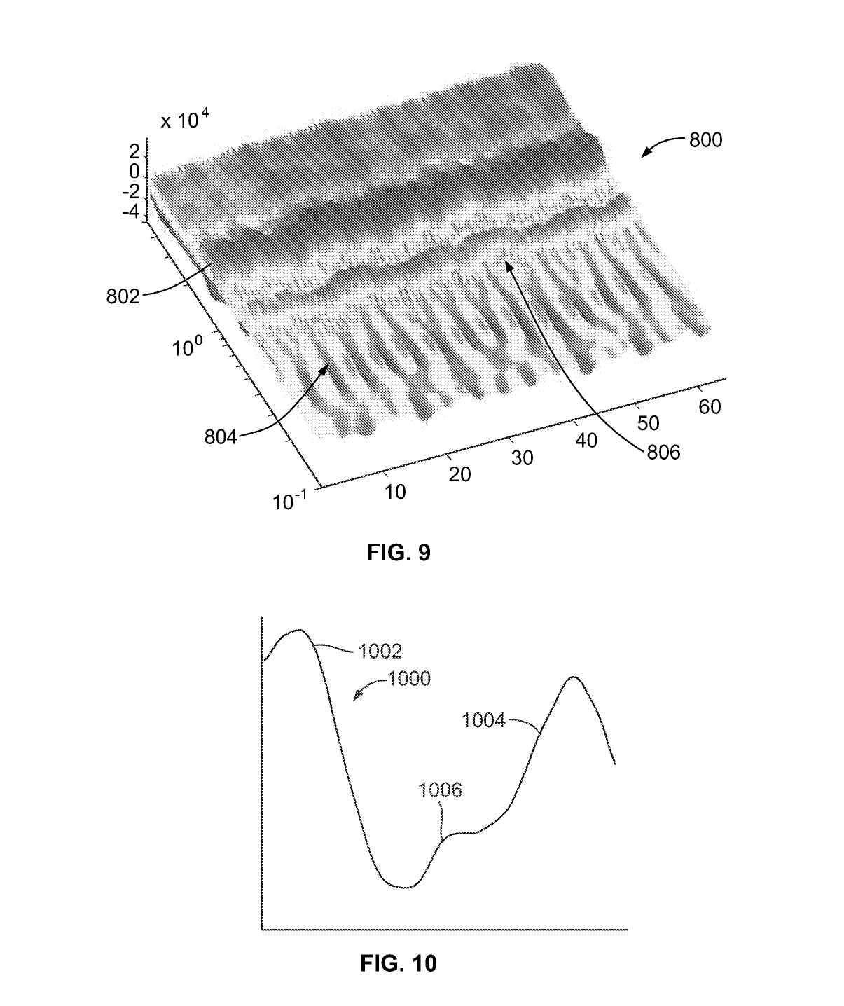

FIG. 8 illustrates a top plan view of a scalogram 800 formed by the synthesized wavelet 700 shown in FIG. 7 transforming the PPG signal 400 shown in FIG. 4, according to an embodiment of the present disclosure. FIG. 9 illustrates an isometric view of the scalogram 800. Referring to FIGS. 4 and 7-9, the scalogram 800 includes a main pulse band 802, respiration components 804, and an additional lower band 806, which is not found in the scalogram 500 (shown in FIGS. 5 and 6). The lower band 806 may represent noise or other interference that is decoupled from the main pulse band 802. Additionally, the lower band 806 may provide additional information that may be analyzed by the physiological parameter determination module 108 (shown in FIG. 1). The lower band 806, by itself or in conjunction with the main pulse band 802 and/or the respiration components 804, may provide a more accurate approximation of the PPG signal 400.

While the synthesized wavelet 700 is described as a double-humped shape, various other mathematical functions and/or shapes may be used to better approximate the shape of the PPG signal 400. In an example, a triple sinusoidal shape may be used. Additionally, other waveforms may be used in addition to, or in place of, the sinusoidal shapes.

FIG. 10 illustrates a simplified PPG signal 1000 over time, according to an embodiment of the present disclosure. The PPG signal 1000 is an example of a physiological signal. However, embodiments of the present disclosure may be used in relation to various other physiological signals, such as blood pressure signals, EKG signals, phonocardiogram signals, ultrasound signals, and the like. Referring to FIGS. 1 and 10, the PPG signal 1000 may be shown as a waveform by a display, such as the display 110, which receives signal data from the physiological signal detection module 102.

The PPG signal 1000 may include a plurality of pulses over a predetermined time period. The time period may be a fixed time period, or the time period may be variable. Moreover, the time period may be a rolling time period, such as a 1-60 second rolling timeframe.

Each pulse may represent a single heartbeat and may include a pulse-transmitted or primary peak 1002 separated from a pulse-reflected or trailing peak 1004 by a dichrotic notch 1006. The primary peak 1002 represents a pressure wave generated from the heart to the point of detection, such as in a finger, forehead, forearm, neck, or the like, where a pulse oximeter sensor, for example, is positioned. The trailing peak 1004 may represent a pressure wave that is reflected from the location proximate to where the pulse oximeter sensor is positioned back toward the heart.

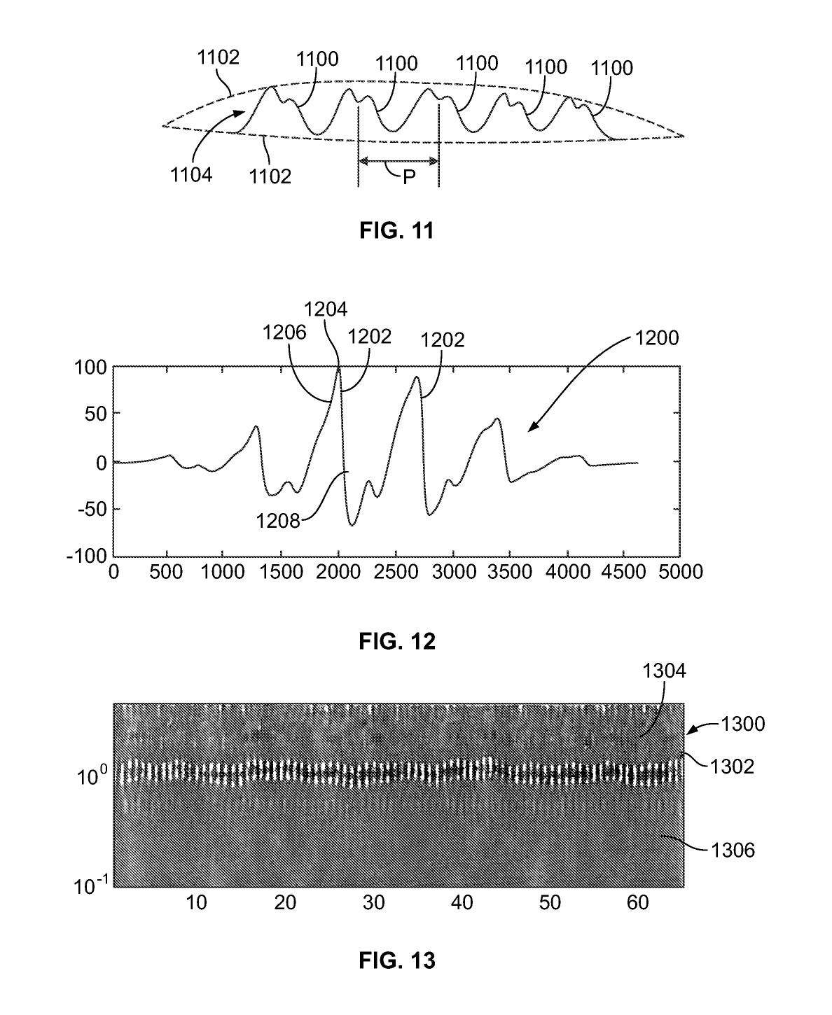

FIG. 11 illustrates a series of PPG signals 1100 within a Gaussian window or envelope 1102, according to embodiment of the present disclosure. The PPG signals 1100 may be part of a PPG signal trace that includes more or less PPG signals 1100 than shown. Referring to FIGS. 1 and 11, the wavelet formation module 104 may select a number of PPG signals 1100 to be contained within the Gaussian window. The wavelet formation module 104 may be programmed to select a certain number of PPG signals 1100 over a predetermined period of time, such as a current 1-60 second rolling timeframe. Alternatively, the wavelet formation module 104 may contain only one PPG signal, such as the PPG signal 1000 shown in FIG. 10, within the Gaussian window. Moreover, the wavelet formation module 104 may change the sampling time over which the Gaussian window 1102 is plotted. For example, the Gaussian window 1102 may occur over changing periods of time, based on detected changes in physiological parameters of an individual.

A natural period p of a PPG waveform 1104 may be attributed to a wavelet scale a. That is, the period p may be equal to the wavelet scale a. In this manner, the period p of the PPG waveform 1104 may be directly linked to the wavelet scale a. In one example, the period p of a cardiac pulsatile component (for example, the heart beat interval) of the PPG waveform 1104 may equal the wavelet scale a. However, the period p and the wavelet scale a may be related in different ways. For example, the period p may be a fraction of the scale a, or vice versa. The Gaussian window or envelope 1102 may be represented by e^{-t*2/2} (where −t*2 is −t²) and has a standard deviation and confines the PPG waveform 1104. By containing the PPG waveform 1104 within the Gaussian window 1102, the wavelet formation module 104 may form a wavelet that is based on the PPG signals 1100. However, the wavelet formation module 104 may form a wavelet based on the PPG signals 1100 through other methods, such as through using the shape of a PPG signal 1100 as one or more peaks of a wavelet, averaging multiple PPG signals 1100 over a certain period time, and using the averaged PPG signal as the shape of one or more peaks of a wavelet, substituting the shape of one or more PPG signals 1100 in place of the peaks of another wavelet, such as the Morlet wavelet 300 shown in FIG. 3, and/or the like.

In general, the wavelet formation module 104 may form a wavelet that is based directly on a physiological signal, such as a PPG signal. The physiological signal may be continuously detected, and the wavelet formation module 104 may adapt the formed wavelet based on changes in the PPG signal over time. Through directly analyzing the PPG signal, the wavelet transform module 104 may shape the wavelet to directly correlate to, conform to, and/or resemble the PPG signal itself.

FIG. 12 illustrates a PPG wavelet 1200, according to an embodiment of the present disclosure. Referring to FIGS. 1 and 12, the wavelet transform module 104 forms the wavelet 1200 through a direct analysis of the PPG signal 400, shown in FIG. 4. That is, the wavelet 1200 is directly formed from and/or based on the PPG signal 400. As shown, the wavelet 1200 may include a plurality of peaks 1202 that are not perfectly smooth and/or sinusoidal in nature. Because the PPG signal 400 may be not a smooth, regular, and even signal, the wavelet 1200 may also not be smooth, regular, and even. Instead, the wavelet 1200 tracks closer to the actual morphology of the PPG signal 400. The wavelet 1200 may include peaks 1202 having sharp points 1204, irregular curves 1206, and linear portions 1208, which are directly correlated to the morphology of the PPG signal 400. Accordingly, the wavelet 1200 may be used with respect to Equation (1) to provide a scalogram that provides accurate, detailed information that is directly related to the actual PPG signal 400 itself.

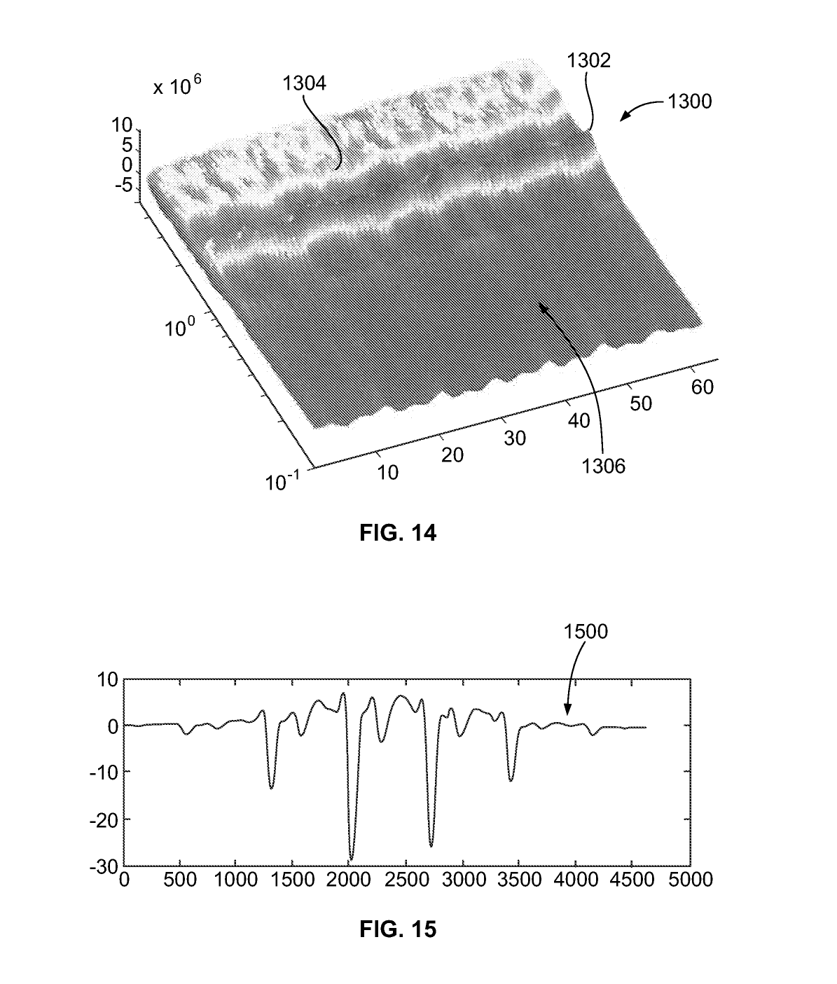

FIG. 13 illustrates a top plan view of a scalogram 1300 formed by the PPG wavelet 1200 shown in FIG. 12 transforming the PPG signal 400 shown in FIG. 4, according to an embodiment of the present disclosure. FIG. 14 illustrates an isometric view of the scalogram 1300. Referring to FIGS. 1 and 13-14, a main pulse band 1302 is separated from components 1304. As an example, the components 1304 may represent additional information that has been separated from the main pulse band 1302. As such, the main pulse band 1302 is clearer, and may be more easily analyzed. Additionally, respiration components 1306 may be reduced in the scalogram, as the components 1306 may not be desired for analysis. In short, by forming the wavelet transform 1200 directly from the PPG signal 400 itself, the resulting scalogram 1300 is clearer and provides information, such as the main pulse band 1302, that may be quickly and reliably analyzed. As an example, unwanted components, noise, and the like may be more clearly decoupled from the main pulse band 1302. The system 100 may therefore provide accurate information regarding a desired physiological parameter, such as pulse rate.

It has been found that the scalogram 1300, which is formed through the wavelet 1200 transforming the PPG signal 400, provides a distinct reduction in respiration components and a distinct reduction in higher characteristic frequency components of the pulse wave. Because the wavelet 1200 is morphology-specific (for example, directly based on the PPG signal 400 itself), the wavelet 1200 causes the scalogram 1300 to suppress components that are not at the main pulse period. As such, the wavelet 1200 may enhance the main pulse band 1302, which may be used for a more accurate analysis and determination of a physiological parameter, such as pulse rate. As an example, the wavelet 1200 may lead to a scalogram in which the amplitude of the main pulse band 1302 is increased, while the amplitudes of the components 1304 and 1306 are decreased.

Alternatively, embodiments of the present disclosure may analyze the components 1304 and 1306 to determine various other physiological parameters, such as respiration rate, blood pressure, and the like. By more clearly decoupling the components 1304 and 1306 from the main pulse band 1302, the system 100 may quickly and readily determine various physiological parameters from the components 1304 and 1306, and the main pulse band 1302. Additionally, it has been found that using the wavelet 1200, which is directly formed from the PPG signal 400, for example, unwanted noise and distortions are filtered from the scalogram 1300, thereby leading to more accurate determinations of physiological parameters.

In an embodiment, the PPG signal 400 may be band pass filtered before the wavelet 1200 is used to transform the PPG signal 400 into the scalogram 1300. As an example, a band pass filter may be used to filter the PPG signal between 0.25 Hz and 10 Hz. In this manner, information from the main pulse band 1302, for example, may be derived without resorting to higher frequency bands for analysis.

FIG. 15 illustrates a PPG wavelet 1500 derived from a derivative of the PPG signal 400 shown in FIG. 4, according to an embodiment of the present disclosure. Embodiments of the present disclosure provide a wavelet that may be directly formed from the PPG signal 400. However, as shown in FIG. 15, the wavelet 1500 may also be derived from a derivative (for example, dx/dt, where x is the PPG signal and t is time) of the PPG signal 400.

As described above, the wavelet formation module 104 (shown in FIG. 1), may be used to form a wavelet based on a physiological signal, such as the PPG signal 400.

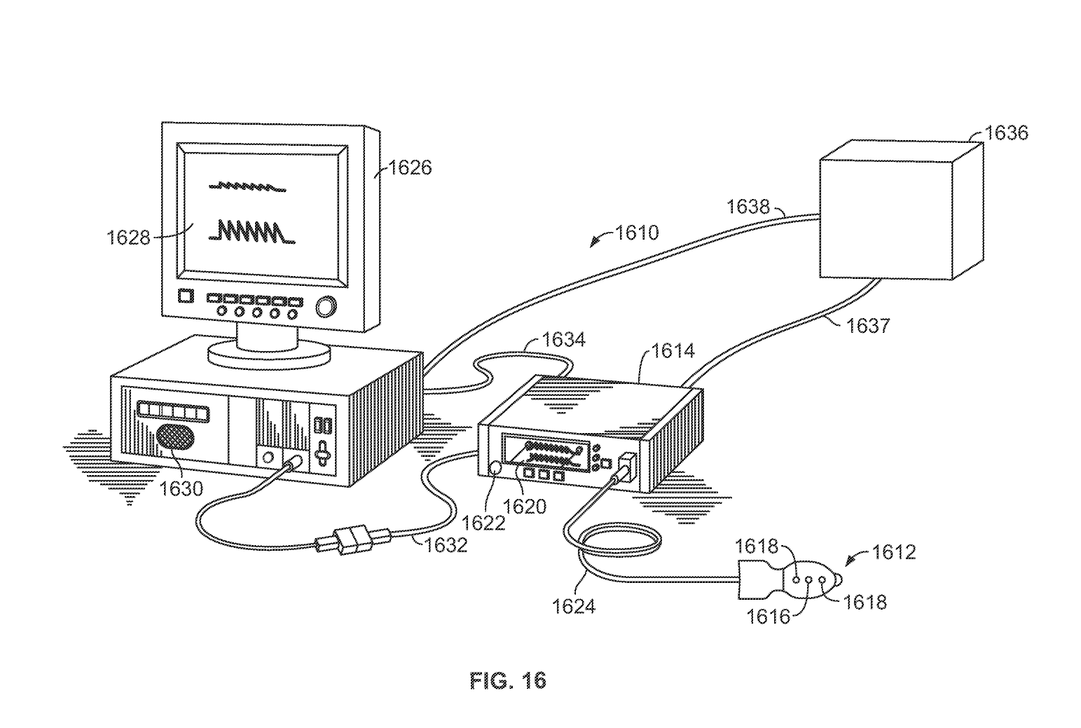

FIG. 16 illustrates an isometric view of a PPG system 1610, according to an embodiment of the present disclosure. The PPG system 1610 may be in communication with, or part of, the system 100, shown in FIG. 1. The PPG system 1610 is an example of a detection sub-system that may be in communication with the physiological signal detection module 102 (shown in FIG. 1). While the system 1610 is shown and described as a PPG system 1610, the system may be various other types of physiological detection systems, such as an electrocardiogram system, a phonocardiogram system, and the like. The PPG system 1610 may be a pulse oximetry system, for example. The system 1610 may include a PPG sensor 1612 and a PPG monitor 1614. The PPG sensor 1612 may include an emitter 1616 configured to emit light into tissue of a patient. For example, the emitter 1616 may be configured to emit light at two or more wavelengths into the tissue of the patient. The PPG sensor 1612 may also include spaced-apart photodetectors 1618 that are configured to detect the emitted light from the emitter 1616 that emanates from the tissue after passing through the tissue. The photodetectors 1618 may be equidistant, but on opposite sides, from the emitter 1616.

The system 1610 may include a plurality of sensors forming a sensor array in place of the PPG sensor 1612. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 1616 and the photodetectors 1618 may be configured to be located on opposite sides of a digit, such as a finger or toe, in which case the light that emanates from the tissue passes completely through the digit. The emitter 1616 and the photodetectors 1618 may be arranged so that light from the emitter 1616 penetrates the tissue and is reflected by the tissue into the detector 1618, such as a sensor designed to obtain pulse oximetry data.

The sensor 1612 or sensor array may be operatively connected to and draw power from the monitor 1614, for example. Optionally, the sensor 1612 may be wirelessly connected to the monitor 1614 and include a battery or similar power supply (not shown). The monitor 1614 may be configured to calculate physiological parameters based at least in part on data received from the sensor 1612 relating to light emission and detection. Alternatively, the calculations may be performed by and within the sensor 1612 and the result of the oximetry reading may be passed to the monitor 1614. Additionally, the monitor 1614 may include a display 1620 configured to display the physiological parameters or other information about the system 1610. The monitor 1614 may also include a speaker 1622 configured to provide an audible sound that may be used in various other embodiments, such as for example, sounding an audible alarm in the event that physiological parameters are outside a predefined normal range.

The sensor 1612, or the sensor array, may be communicatively coupled to the monitor 1614 via a cable 1624. Alternatively, a wireless transmission device (not shown) or the like may be used instead of, or in addition to, the cable 1624.

The system 1610 may also include a multi-parameter workstation 1626 operatively connected to the monitor 1614. The workstation 1626 may be or include a computing sub-system 1630, such as standard computer hardware. The computing sub-system 1630 may include one or more modules and control units, such as processing devices that may include one or more microprocessors, microcontrollers, integrated circuits, memory, such as read-only and/or random access memory, and the like. The workstation 1626 may include a display 1628, such as a cathode ray tube display, a flat panel display, such as a liquid crystal display (LCD), a light-emitting diode (LED) display, a plasma display, or any other type of monitor. The computing sub-system 1630 of the workstation 1626 may be configured to calculate physiological parameters and to show information from the monitor 1614 and from other medical monitoring devices or systems (not shown) on the display 1628. For example, the workstation 1626 may be configured to display an estimate of a patient's blood oxygen saturation generated by the monitor 1614 (referred to as an SpO₂ measurement), pulse rate information from the monitor 1614, and blood pressure from a blood pressure monitor (not shown) on the display 1628.

The monitor 1614 may be communicatively coupled to the workstation 1626 via a cable 1632 and/or 1634 that is coupled to a sensor input port or a digital communications port, respectively and/or may communicate wirelessly with the workstation 1626. Additionally, the monitor 1614 and/or workstation 1626 may be coupled to a network to enable the sharing of information with servers or other workstations. The monitor 1614 may be powered by a battery or by a conventional power source such as a wall outlet.

The system 1610 may also include a fluid delivery device 1636 that is configured to deliver fluid to a patient. The fluid delivery device 1636 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 1636 may be configured to adjust the quantity or concentration of fluid delivered to a patient.

The fluid delivery device 1636 may be communicatively coupled to the monitor 1614 via a cable 1637 that is coupled to a digital communications port or may communicate wirelessly with the workstation 1626. Alternatively, or additionally, the fluid delivery device 1636 may be communicatively coupled to the workstation 1626 via a cable 1638 that is coupled to a digital communications port or may communicate wirelessly with the workstation 1626.

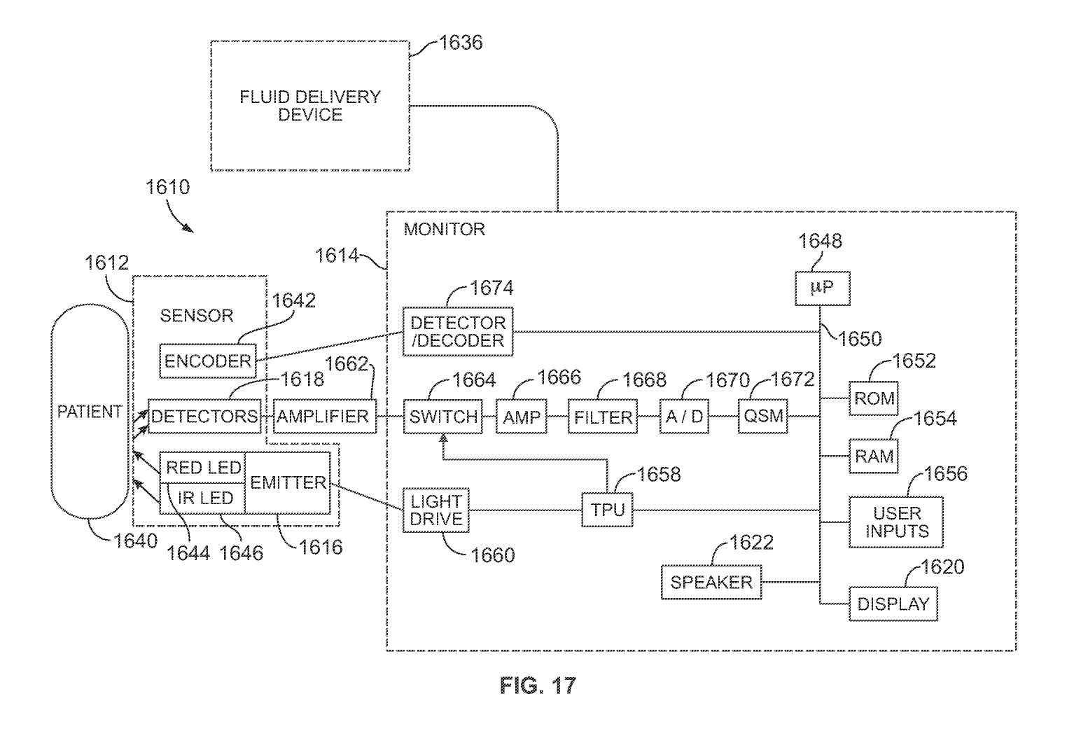

FIG. 17 illustrates a simplified block diagram of the PPG system 1610, according to an embodiment of the present disclosure. When the PPG system 1610 is a pulse oximetry system, the emitter 1616 may be configured to emit at least two wavelengths of light (for example, red and infrared) into tissue 1640 of a patient. Accordingly, the emitter 1616 may include a red light-emitting light source such as a red light-emitting diode (LED) 1644 and an infrared light-emitting light source such as an infrared LED 1646 for emitting light into the tissue 1640 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG system 1610 is described in terms of a pulse oximetry system. However, the PPG system 1610 may be various other types of systems. For example, the PPG system 1610 may be configured to emit more or less than two wavelengths of light into the tissue 1640 of the patient. Further, the PPG system 1610 may be configured to emit wavelengths of light other than red and infrared into the tissue 1640. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 1610. The photodetectors 1618 may be configured to be specifically sensitive to the chosen targeted energy spectrum of the emitter 1616.

The photodetectors 1618 may be configured to detect the intensity of light at the red and infrared wavelengths. Alternatively, each sensor in the array may be configured to detect an intensity of a single wavelength. In operation, light may enter the photodetectors 1618 after passing through the tissue 1640. The photodetectors 1618 may convert the intensity of the received light into electrical signals. The light intensity may be directly related to the absorbance and/or reflectance of light in the tissue 1640. For example, when more light at a certain wavelength is absorbed or reflected, less light of that wavelength is received from the tissue by the photodetectors 1618. After converting the received light to an electrical signal, the photodetectors 1618 may send the signal to the monitor 1614, which calculates physiological parameters based on the absorption of the red and infrared wavelengths in the tissue 1640.

In an embodiment, an encoder 1642 may store information about the sensor 1612, such as sensor type (for example, whether the sensor is intended for placement on a forehead or digit) and the wavelengths of light emitted by the emitter 1616. The stored information may be used by the monitor 1614 to select appropriate algorithms, lookup tables and/or calibration coefficients stored in the monitor 1614 for calculating physiological parameters of a patient. The encoder 1642 may store or otherwise contain information specific to a patient, such as, for example, the patient's age, weight, diagnosis, and/or the like. The information may allow the monitor 1614 to determine, for example, patient-specific threshold ranges related to the patient's physiological parameter measurements, and to enable or disable additional physiological parameter algorithms. The encoder 1642 may, for instance, be a coded resistor that stores values corresponding to the type of sensor 1612 or the types of each sensor in the sensor array, the wavelengths of light emitted by emitter 1616 on each sensor of the sensor array, and/or the patient's characteristics. Optionally, the encoder 1642 may include a memory in which one or more of the following may be stored for communication to the monitor 1614: the type of the sensor 1612, the wavelengths of light emitted by emitter 1616, the particular wavelength each sensor in the sensor array is monitoring, a signal threshold for each sensor in the sensor array, any other suitable information, or any combination thereof.

Signals from the photodetectors 1618 and the encoder 1642 may be transmitted to the monitor 1614. The monitor 1614 may include a general-purpose control unit, such as a microprocessor 1648 connected to an internal bus 1650. The microprocessor 1648 may be configured to execute software, which may include an operating system and one or more applications, as part of performing the functions described herein. A read-only memory (ROM) 1652, a random access memory (RAM) 1654, user inputs 1656, the display 1620, and the speaker 1622 may also be operatively connected to the bus 1650.

The RAM 1654 and the ROM 1652 are illustrated by way of example, and not limitation. Any suitable computer-readable media may be used in the system for data storage. Computer-readable media are configured to store information that may be interpreted by the microprocessor 1648. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 1614 may also include a time processing unit (TPU) 1658 configured to provide timing control signals to a light drive circuitry 1660, which may control when the emitter 1616 is illuminated and multiplexed timing for the red LED 1644 and the infrared LED 1646. The TPU 1658 may also control the gating-in of signals from the photodetectors 1618 through an amplifier 1662 and a switching circuit 1664. The signals are sampled at the proper time, depending upon which light source is illuminated. The received signals from the photodetectors 1618 may be passed through an amplifier 1666, a low pass filter 1668, and an analog-to-digital converter 1670. The digital data may then be stored in a queued serial module (QSM) 1672 (or buffer) for later downloading to RAM 1654 as QSM 1672 fills up. In an embodiment, there may be multiple separate parallel paths having amplifier 1666, filter 1668, and A/D converter 1670 for multiple light wavelengths or spectra received.

The microprocessor 1648 may be configured to determine the patient's physiological parameters, such as SpO₂ and pulse rate using various algorithms and/or look-up tables based on the value(s) of the received signals and/or data corresponding to the light received by the photodetectors 1618. The signals corresponding to information about a patient, and regarding the intensity of light emanating from the tissue 1640 over time, may be transmitted from the encoder 1642 to a decoder 1674. The transmitted signals may include, for example, encoded information relating to patient characteristics. The decoder 1674 may translate the signals to enable the microprocessor 1648 to determine the thresholds based on algorithms or look-up tables stored in the ROM 1652. The user inputs 1656 may be used to enter information about the patient, such as age, weight, height, diagnosis, medications, treatments, and so forth. The display 1620 may show a list of values that may generally apply to the patient, such as, for example, age ranges or medication families, which the user may select using the user inputs 1656.

The fluid delivery device 1636 may be communicatively coupled to the monitor 1614. The microprocessor 1648 may determine the patient's physiological parameters, such as a change or level of fluid responsiveness, and display the parameters on the display 1620. In an embodiment, the parameters determined by the microprocessor 1648 or otherwise by the monitor 1614 may be used to adjust the fluid delivered to the patient via fluid delivery device 1636.

As noted, the PPG system 1610 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters measure and display various blood flow characteristics including, but not limited to, the oxygen saturation of hemoglobin in arterial blood.

A pulse oximeter may include a light sensor, similar to the sensor 1612, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as the PPG signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 1610 and pulse oximetry may be further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

It will be understood that the present disclosure is applicable to any suitable physiological signals and that PPG are used for illustrative purposes. Those skilled in the art will recognize that the present disclosure has wide applicability to other signals including, but not limited to other physiological signals (for example, electrocardiogram, electroencephalogram, electrogastrogram, electromyogram, heart rate signals, pathological sounds, ultrasound, or any other suitable biosignal) and/or any other suitable signal, and/or any combination thereof. Thus, the wavelet formation module 104 (shown in FIG. 1) may be used to form a wavelet based on various other physiological signals, such as a blood pressure signal, an EKG signal, and the like.

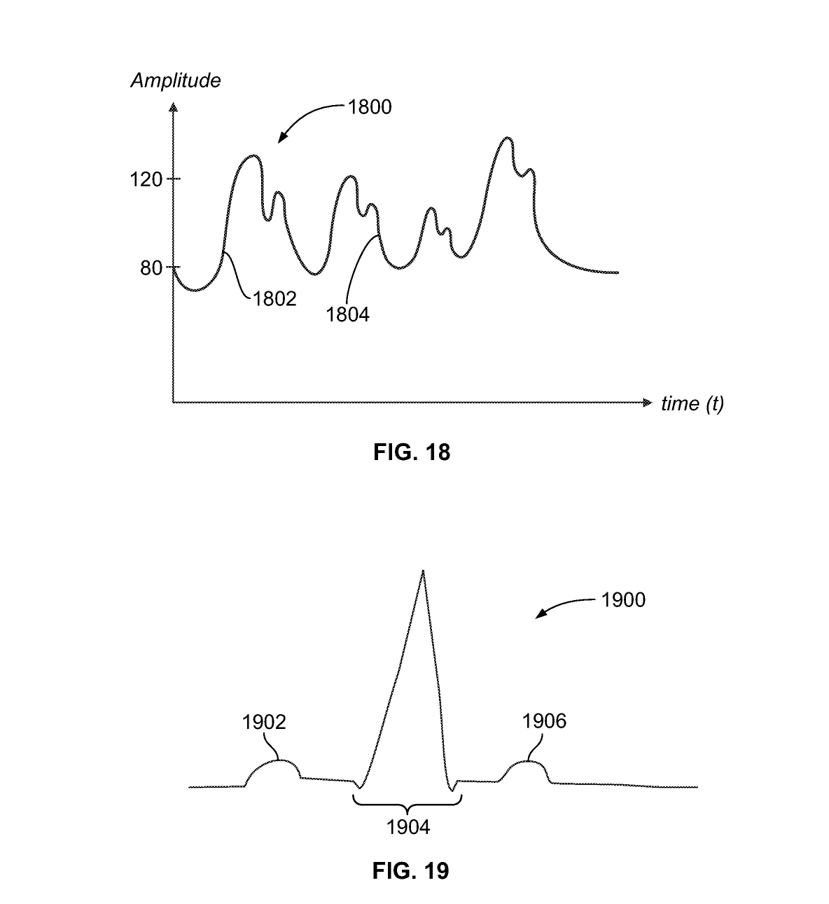

FIG. 18 illustrates a blood pressure signal 1800 over time, according to an embodiment of the present disclosure. Blood pressure represents a measurement that quantifies a pressure exerted by circulating blood upon walls of blood vessels. In general, blood pressure is an example of a principal vital sign. Typically, blood pressure may be measured through use of a sphygmomanometer, or blood pressure cuff, and a stethoscope. However, blood pressure may also be invasively detected through an arterial line catheter, for example.

The blood pressure signal 1800 is an example of a physiological signal that may be detected by a detection sub-system, such as a blood pressure detection sub-system. As shown, an amplitude of the blood pressure signal 1800, may vary over time. For example, the amplitude may vary with respect to a base, average, or mean blood pressure of 120 systolic over 80 diastolic. As an example, the amplitude may change from blood pressure pulse 1802 to blood pressure pulse 1804. The physiological signal detection module 102 (shown in FIG. 1) and/or a pressure detection sub-system, may track the change in amplitude of the blood pressure signal 1800, and store the change in amplitude for analysis. The wavelet formation module 104 (shown in FIG. 1) may form a wavelet based on the blood pressure signal 1800, for example. In an embodiment, the wavelet formation module 104 may contain the blood pressure signal 1800 within a Gaussian window, as described above with respect to FIG. 11, to form the wavelet.

FIG. 19 illustrates an EKG signal 1900, according to an embodiment of the present disclosure. Electrocardiography represents a transthoracic (across the thorax or chest) measurement of electrical activity of the heart over a period of time, as detected by electrodes attached to the outer surface of the skin and recorded by a device external to the body. The recording produced by the noninvasive procedure is termed an EKG or ECG. An EKG is used to measure the rate and regularity of heartbeats, as well as the size and position of the chambers, the presence of any damage to the heart, and the effects of drugs or devices used to regulate the heart, such as a pacemaker.

The EKG signal 1900 is an example of a physiological signal. The EKG signal 1900 includes a P-wave 1902, a QRS complex 1904, and a T wave 1906. The P wave 1902 indicates atrial depolarization, or contraction of the atrium. The QRS complex 1904 indicates ventricular depolarization, or contraction of the ventricles. The T wave 1906 indicates ventricular repolarization.

The wavelet formation module 104 (shown in FIG. 1) may form a wavelet based on the EKG signal 1900, for example. In an embodiment, the wavelet formation module 104 may contain the EKG signal 1900 within a Gaussian window, as described above with respect to FIG. 11, to form the wavelet.

While physiological signals, such as PPG signals, blood pressure signals, and EKG signals are shown and described, it is understood that embodiments of the present disclosure may be used with respect to various other physiological signals.

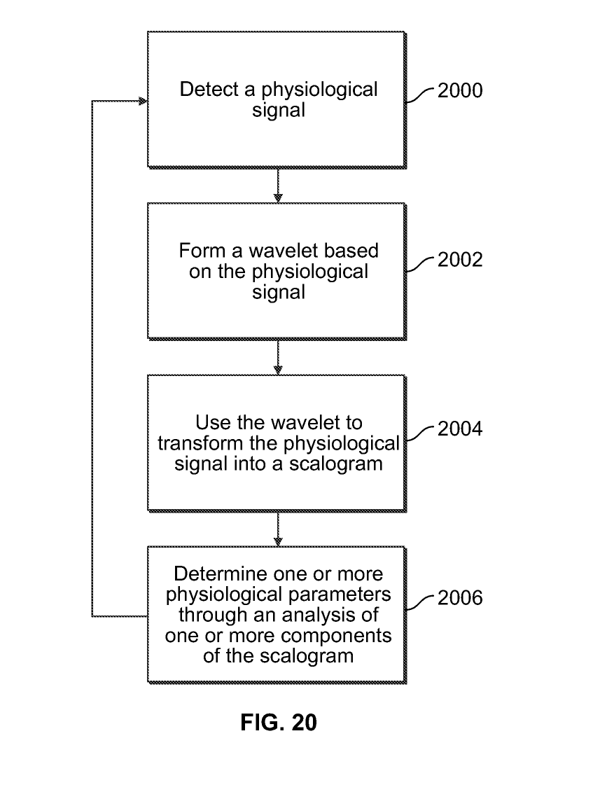

FIG. 20 illustrates a flow chart of a method of determining one or more physiological parameters, according to an embodiment of the present disclosure. The process begins at 2000, in which a physiological signal, such as any of those described above, is detected. Next, at 2002, a wavelet based on the physiological signal is formed. The wavelet may be directly based on the physiological signal, such as through one or more portions or traces of the physiological being analyzed to form the wavelet having an analogous or similar shape to the physiological signal. Alternatively, the wavelet may be synthesized, such as through shapes, forms, and contours that are analogous or otherwise similar to a shape of a normal physiological signal, for example.

Then, at 2004, the wavelet is applied to the physiological signal to transform the physiological signal into a scalogram. Accordingly, the scalogram is highly correlated to the physiological signal, as the transforming wavelet is based on the physiological signal (in contrast to a generalized waveform that is not related to the physiological signal). Then, at 2006, one or more physiological parameters are determined through an analysis of one or more components of the scalogram. It has been found that a scalogram formed by the physiological signal-based wavelet operating on the physiological signal provides a scalogram with distinctly decoupled component parts, such as a pulse band and respiratory components, that may be readily and accurately analyzed.

After 2006, the process returns to 2000. Accordingly, the wavelet may be continually adapted and reformed based on the current state of a physiological signal. As such, the embodiments of the present disclosure provide a system and method that may continually adapt to changing physiological conditions of an individual that is being monitored through one or more detection sub-systems.

Embodiments of the present disclosure provide a system and method that forms a wavelet that may be described by a real function. That is, the wavelet may include no complex parts. Alternatively, the wavelet may be described by real and complex, including imaginary, components. For example, in an embodiment, a negative part of a spectrum of a wavelet may be removed, thereby producing a complex wavelet that may be analyzed. Such an embodiment may be particularly useful when analyzing non-stationary signals with changing component features and variable natural periods.

Embodiments of the present disclosure may be used to determine physiological parameters, such as pulse rate. Additionally, embodiments of the present disclosure may be used to determine various other physiological parameters, such as respiration rate, respiration effort, changes in blood pressure, cardiac output, vasoconstriction, awareness and arousal, and the like.

Embodiments of the present disclosure provide a system and method of forming a wavelet that may be based on a physiological signal itself. Alternatively, the wavelet may be based on a shape that conforms to a shape of a general physiological signal. Because the wavelet is based on the physiological signal itself (whether through direct analysis of the PPG signal and formation of the wavelet based on the direct analysis of the PPG signal, or through a shape that correlates to a general shape of the physiological signal), the resulting scalogram provides clear, reliable, accurate, and useful information that may be used to determine one or more physiological parameters.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “module.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35U.S.C. §112, sixth paragraph, unless and until such claim limitations expressly use the phrase “means for” followed by a statement of function void of further structure.

What is claimed is:

1. A system for analyzing a physiological signal detected from an individual, the system comprising:
a physiological signal detection module configured to detect the physiological signal of the individual;
a wavelet formation module configured to form a wavelet based on the physiological signal; and
a wavelet transform module configured to generate a scalogram by transforming the physiological signal with the wavelet based on the physiological signal.

2. The system of claim 1, further comprising a physiological parameter determination module configured to determine one or more physiological parameters of the individual through an analysis of the scalogram.

3. The system of claim 1, further comprising a detection sub-system in communication with the physiological signal detection module, wherein the physiological signal detection module is configured to receive the physiological signal from the detection sub-system.

4. The system of claim 3, wherein the detection sub-system comprises a photoplethysmograph (PPG) sub-system, and wherein the physiological signal comprises a PPG signal.

5. The system of claim 3, wherein the detection sub-system comprises a blood pressure detection sub-system, and wherein the physiological signal comprises a blood pressure signal.

6. The system of claim 3, wherein the detection sub-system comprises an electrocardiogram (EKG) sub-system, and wherein the physiological signal comprises an EKG signal.

7. The system of claim 1, wherein the wavelet formation module is configured to form the wavelet by directly analyzing the physiological signal and shaping the wavelet to directly correlate to the physiological signal.

8. The system of claim 7, wherein the wavelet formation module is configured to contain at least a portion of the physiological signal within a Gaussian window to form the wavelet.

9. The system of claim 1, wherein the wavelet is a derivative of the physiological signal.

10. The system of claim 1, wherein the wavelet formation module is configured to form the wavelet by using one or more shapes that are similar to one or more portions of a generic shape of the physiological signal.

11. A method of analyzing a physiological signal detected from an individual, the method comprising:
detecting a physiological signal of the individual with a physiological signal detection module;
forming a wavelet based on the physiological signal with a wavelet formation module; and
generating a scalogram with a wavelet transform module by transforming the physiological signal with the wavelet based on the physiological signal.

12. The method of claim 11, wherein the physiological signal comprises one or more of a photoplethysmograph (PPG) signal, a blood pressure signal, or an electrocardiogram (EKG) signal.

13. The method of claim 11, wherein the forming operation comprises directly analyzing the physiological signal and shaping the wavelet to directly correlate to the physiological signal.

14. The method of claim 13, wherein the forming operation comprises containing at least a portion of the physiological signal within a Gaussian window to form the wavelet.

15. The method of claim 13, wherein the forming operation comprises using one or more shapes that are similar to one or more portions of a generic shape of the physiological signal.

16. A tangible and non-transitory computer readable medium that includes one or more sets of instructions configured to direct a computer to:
detect a physiological signal of an individual;
form a wavelet based on the physiological signal; and
generate a scalogram by transforming the physiological signal with the wavelet based on the physiological signal.

17. The tangible and non-transitory computer readable medium of claim 16, wherein the physiological signal comprises one or more of a photoplethysmograph (PPG) signal, a blood pressure signal, or an electrocardiogram (EKG) signal.

18. The tangible and non-transitory computer readable medium of claim 16, wherein the one or more instructions are further configured to direct the computer to directly analyze the physiological signal and shape the wavelet to directly correlate to the physiological signal.

19. The tangible and non-transitory computer readable medium of claim 18, wherein the one or more instructions are further configured to direct the computer to contain at least a portion of the physiological signal within a Gaussian window.

20. The tangible and non-transitory computer readable medium of claim 16, wherein the one or more instructions are further configured to direct the computer to use one or more shapes that are similar to one or more portions of a generic shape of the physiological signal to form the wavelet.
